(19) United States
(12) Patent Application Publication
(10) Pub. No.: US 2026/0297624 A1
(43) Pub. Date: Oct. 1, 2026

(54) RECOMBINANT ENZYME FOR THE ACCURATE INSERTION OF DNA SEQUENCES IN EUKARYOTIC CELLS

(71) Applicant: Rensselaer Polytechnic Institute, Troy, NY (US)

(72) Inventors: WILLIAM G. LAWLER, Troy, NY (US); MATTHEOS AG KOFFAS, Niskayuna, NY (US)

(73) Assignee: Rensselaer Polytechnic Institute, Troy, NY (US)

(21) Appl. No.: 19/479,159

(22) PCT Filed: Apr. 29, 2024
(86) PCT No.: PCT/US2024/026762

§ 371 Date: Oct. 27, 2025

Related U.S. Application Data
(60) Provisional application No. 63/462,377, filed on Apr. 27, 2023.
(60) Provisional application No. 63/639,007, filed on Apr. 26, 2024.

(51) Int. Cl. C12N 15/90; C12N 9/12; C12N 15/86

(52) U.S. Cl. CPC C12N 15/90; C12N 9/1276; C12N 15/86; C07K 2319/80; C12N 2740/16043; C12N 2740/16222

(57) ABSTRACT

A construct for site-specific insertion of DNA fragments and transgenes into a genome includes a recombinant enzyme. The enzyme includes a modified HIV-1 integrase (HIV IN) tetramer having a plurality of mutations to favor a heterodimer of heterodimers construction with first and second outer monomers bound to first and second inner monomers, respectively. Transcription Activator Like Effectors (TALEs) are bound to N-terminal regions of the outer monomers via polypeptide linkers. The two TALEs grant the enzyme sequence specificity and prevent non-specific interactions with a target DNA structure. The modified HIV IN monomers ensure binding of TALEs to the outer-most monomers and arrangement of the TALEs relative to the HIV IN's catalytic region. Polynucleotides are bound to inner monomers of the HIV IN. Upon transport to the nucleus, either alone or within a lentiviral capsid, the TALEs interact with the DNA to localize the HIV IN, which then integrates the polynucleotides.

### CROSS-REFERENCE TO RELATED APPLICATIONS

This application claims the benefit of U.S. Provisional Patent Application Nos. 63/639,007, filed Apr. 26, 2024, and 63/462,377, filed Apr. 27, 2023, which are incorporated by reference as if disclosed herein in their entireties.

### BACKGROUND

In the field of genomics, there are a variety of mechanisms that have evolved naturally to insert transgenes and exogenous DNA fragments into an organism's genome to accomplish particular goals. Many of the mechanisms of these enzymes have been elucidated, and their goal is transposition of genetic material into new locations in the genome. Unfortunately, many of these enzymes are from transposons or viruses, which do not support the longevity of the cell, nor the stability of its genome. Because of this, many of the mechanisms in nature are not accurate enough to reliably insert DNA into specific gene loci with virtually no off-target mutations. While there are other mechanisms such as different recombinases, these are also limited in their ability to insert DNA because they require target sequences to already be present in the genome to perform insertions, which usually requires an initial insertion to be performed.

Attempts have been made to alter certain proteins to increase their sequence specificity for DNA insertion, but for the most part, these have not shown the accuracy necessary to make them useful for many clinical applications, or they are not able to transfect cells effectively in vivo. In recent years, methods have been developed to insert, edit, or remove small regions of DNA on the scale of less than a hundred nucleotides, such as prime editors, twin prime-editors, and base editors. However, almost all of these methods rely in some way on the same Cas backbone, and are prone to similar issues with regards to accuracy. Even “highly-accurate” variations like Sniper-Cas9 still exhibit off-target mutation rates as high as 10% in some cases, with other variations displaying a distinct tradeoff between increasing accuracy and maintaining the efficiency of modification.

Because of the difficulty in accurately editing genomic material and high risk of off-target alterations, particularly in vivo, current gene therapy treatments (especially those involving forms of CRISPR or its derivatives) are relegated to the most severe cases and require cells to be extracted before they can be modified, minimizing the overall usefulness of these treatments. What is desired, therefore, are gene editing systems and methods that enable site-specific, in vivo genome editing and, thus, reliable gene therapy treatments for patients with genetic abnormalities.

### SUMMARY

Aspects of the present disclosure are directed to a recombinant enzymatic gene editing system. In some embodiments, the recombinant enzymatic gene editing system includes a recombinant enzyme including an HIV integrase (HIV IN) and two or more Transcription Activator Like Effectors (TALEs) bound to the HIV IN. In some embodiments, the recombinant enzyme includes one or more polynucleotides bound to the HIV IN to form a gene editing construct. In some embodiments, the gene editing construct is provided in a lentiviral capsid, as part of a lentiviral vector, or combinations thereof.

In some embodiments, the HIV IN includes four monomeric subunits including a first inner monomer and a second inner monomer and a first outer monomer and a second outer monomer. In some embodiments, at least two of the monomeric subunits have a TALE bound to N-terminal domains thereof. In some embodiments, a first TALE is bound to the first outer monomer of the HIV IN by a first polypeptide linker, and a second TALE is bound to the second outer monomer of the HIV IN by a second polypeptide linker. In some embodiments, the polypeptide linkers are rigid. In some embodiments, the polypeptide linkers are flexible. In some embodiments, the polypeptide linkers are composed of glycine residues. In some embodiments, the polypeptide linkers include repeats of GGGS domains, EAAAK domains, or combinations thereof. In some embodiments, the polypeptide linkers include alternating repeats of GGGS domains, EAAAK domains, or combinations thereof. In some embodiments, the polypeptide linkers include GGGS GGGS GGGS GGGS (SEQ. ID NO.: 5). In some embodiments, the first polypeptide linker and the second polypeptide linker are composed of glycine residues. In some embodiments, the first TALE and the second TALE are of differing lengths. In some embodiments, the first TALE is longer than the second TALE. In some embodiments, the first TALE is configured to bind a target DNA structure at a first region and the second TALE is configured to bind the target DNA structure at a second region, wherein the first region and the second region are spaced apart on the target DNA structure by between about 15 base pairs and about 25 base pairs. In some embodiments, the HIV IN includes a wild-type HIV IN having a mutation to reduce HIV IN association with LEDGF/p75; E152Q mutations to each of the first outer monomer and the second outer monomer; K186Q mutations to each of the first outer monomer and the second outer monomer, or combinations thereof.

Aspects of the present disclosure are directed to a method of editing genomic material in a target organism, e.g., a patient. In some embodiments, the method includes providing a recombinant enzyme; binding one or more polynucleotides to the recombinant enzyme to form a gene editing construct administering an effective amount of the gene editing construct to the patient; transporting a concentration of the gene editing construct to a cellular nucleus in the patient; localizing the gene editing construct on a target DNA structure via the TALEs; and inserting the one or more polynucleotides into the genomic material of the patient at the target DNA structure.

In some embodiments, the recombinant enzyme includes an HIV IN tetramer and two or more TALEs bound to the HIV IN tetramer. In some embodiments, the HIV IN is designed to include four monomeric subunits including a first inner monomer and a second inner monomer and a first outer monomer and a second outer monomer. In some embodiments, the inner monomers and/or the outer monomers include mutations to preferentially bind in a desired pattern, to increase the likelihood of correctly assembling the desired tetrameric structure. In some embodiments, at least two of the monomeric subunits have a TALE bound to N-terminal domains thereof. In some embodiments, a first TALE is bound to a first outer monomer of the HIV IN by a first polypeptide linker, and a second TALE is bound to a second outer monomer of the HIV IN by a second polypeptide linker. In some embodiments, the polypeptide linkers are rigid. In some embodiments, the polypeptide linkers are flexible. In some embodiments, the polypeptide linkers are composed of glycine residues. In some embodiments, the polypeptide linkers include repeats of GGGS domains, EAAAK domains, or combinations thereof. In some embodiments, the polypeptide linkers include alternating repeats of GGGS domains, EAAAK domains, or combinations thereof. In some embodiments, the polypeptide linkers include GGGS GGGS GGGS GGGS (SEQ. ID NO.: 5). In some embodiments, the first polypeptide linker and the second polypeptide linker are composed of glycine residues. In some embodiments, the first TALE and the second TALE are of differing lengths. In some embodiments, the first TALE is longer than the second TALE. In some embodiments, the first TALE is configured to bind the target DNA structure at a first region and the second TALE is configured to bind the target DNA structure at a second region, wherein the first region and the second region are spaced apart on the target DNA structure by between about 15 base pairs and about 25 base pairs. In some embodiments, the gene editing construct is provided in a lentiviral capsid, as part of a lentiviral vector, or combinations thereof. In some embodiments, the HIV IN includes a wild-type HIV IN having a mutation to reduce HIV IN association with LEDGF/p75; E152Q mutations to each of the first outer monomer and the second outer monomer; K186Q mutations to each of the first outer monomer and the second outer monomer, or combinations thereof.

Aspects of the present disclosure are directed to a recombinant enzymatic gene editing system including a recombinant enzyme. In some embodiments, the recombinant enzyme includes an HIV integrase (HIV IN) tetramer including a first inner monomer and a second inner monomer and a first outer monomer and a second outer monomer, wherein the first inner monomer and the second inner monomer are bound to each other and the first outer monomer is bound to the first inner monomer and the second outer monomer is bound to the second inner monomer; and a first TALE bound to an N-terminal region of the first outer monomer via a first polypeptide linker and a second TALE bound to an N-terminal region of the second outer monomer via a second polypeptide linker. In some embodiments, the one or more polynucleotides bound to at least one of the first inner monomer and the second inner monomer to form a gene editing construct. In some embodiments, a lentiviral capsid encapsulates the gene editing construct.

In some embodiments, the polypeptide linkers are rigid. In some embodiments, the polypeptide linkers are flexible. In some embodiments, the polypeptide linkers are composed of glycine residues. In some embodiments, the polypeptide linkers include repeats of GGGS domains, EAAAK domains, or combinations thereof. In some embodiments, the polypeptide linkers include alternating repeats of GGGS domains, EAAAK domains, or combinations thereof. In some embodiments, the polypeptide linkers include GGGS GGGS GGGS GGGS (SEQ. ID NO.: 5). In some embodiments, the first polypeptide linker and the second polypeptide linker are composed of glycine residues. In some embodiments, the first TALE and the second TALE are of differing lengths. In some embodiments, the first TALE is longer than the second TALE. In some embodiments, each TALE targets a DNA structure segment between about 8 base pairs and about 31 base pairs. In some embodiments, the first TALE targets a DNA structure segment of about 30 base pairs. In some embodiments, the HIV IN tetramer includes a wild-type HIV IN having the following mutations: for the first inner monomer, Y99D, K103E, K173E, and K186E; for the second inner monomer, E96K, Y99E, K103E, V201H, D25K, and E11K; for the first outer monomer, E87K, E96K, E152Q, K186Q, and K215E; for the second outer monomer, E152Q, K173E, T174K, K186Q, and I204D; and a mutation to reduce HIV IN association with LEDGF/p75. In some embodiments, the HIV IN tetramer includes a wild-type HIV IN having the following mutations: for the first inner monomer, Y99D, K103E, K173E, and K186E; for the second inner monomer, E96K, Y99E, K103E, V201H, D25K, and E11K; for the first outer monomer, E87K, E96K, E152Q, K186Q, and K215E; for the second outer monomer, E152Q, K173E, T174K, K186Q, and I204D; a mutation to reduce HIV IN association with LEDGF/p75; and functional equivalents thereof.

### BRIEF DESCRIPTION OF THE DRAWINGS

The drawings show embodiments of the disclosed subject matter for the purpose of illustrating the invention. However, it should be understood that the present application is not limited to the precise arrangements and instrumentalities shown in the drawings, wherein:

FIG. 1 is a schematic representation of a recombinant enzymatic gene editing system according to some embodiments of the present disclosure;

FIGS. 2A-2D are protein structures of modified monomeric subunits in a HIV integrase (HIV IN) according to some embodiments of the present disclosure;

FIG. 2E is a graph showing simulated contact energy data between modified monomeric subunits in a HIV IN according to some embodiments of the present disclosure;

FIGS. 3A-3B are images of Western blots of HIV IN dimers according to some embodiments of the present disclosure; and

FIG. 4 is a chart of a method of editing genomic material in a target organism, e.g., a human patient, according to some embodiments of the present disclosure.

### DETAILED DESCRIPTION

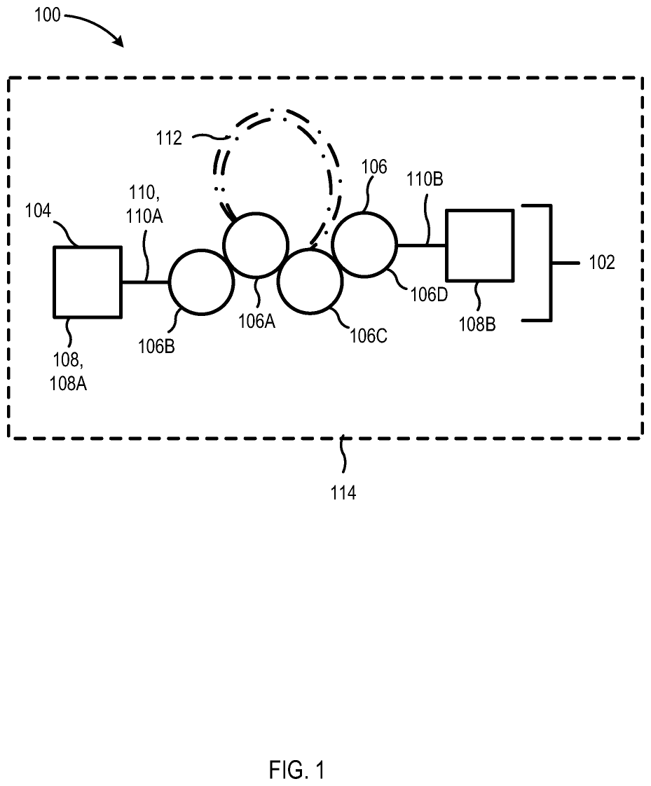

Referring now to FIG. 1, some embodiments of the present disclosure are directed to a recombinant enzymatic gene editing system 100. In some embodiments, system 100 includes a gene editing construct 102. Gene editing construct 102 is designed and configured to transport desired polynucleotides, also referred to herein as “transgenes” or “DNA fragments,” from an extracellular environment to a cellular nuclear environment, and further insert the polynucleotide into a genome with high accuracy, as will be discussed in greater detail below. As discussed herein, embodiments of the present disclosure refer to the insertion of DNA fragments into the genome of a target organism, e.g., in a clinical setting, in vivo, etc., at a targeted genomic location, however the present disclosure is not intended to be limiting in this regard, as the genomic editing by gene editing construct 102 can be accomplished in in vitro or otherwise ex vivo settings as well, e.g., editing of individual cells or a plurality of cells in vitro. In some embodiments, target organism includes any eukaryote, including one or more cells thereof. In some embodiments, the target organism is a human patient. In some embodiments, the insertion of DNA fragments has a therapeutic effect on the target organism/cell. In some embodiments, the insertion of DNA fragments yields increased production by the target organism/cell of one or more desired compounds, e.g., increased chemical production.

Still referring to FIG. 1, gene editing construct 102 includes a recombinant enzyme 104. In some embodiments, recombinant enzyme 104 includes an HIV integrase (HIV IN) 106. In some embodiments, HIV IN 106 is a tetrameter including four monomeric subunits including a first inner monomer 106A and a second inner monomer 106C, and a first outer monomer 106B and a second outer monomer 106D. In some embodiments, first inner monomer 106A and second inner monomer 106C are bound to each other. In some embodiments, first outer monomer 106B is bound to first inner monomer 106A. In some embodiments, second outer monomer 106D is bound to second inner monomer 106C. In some embodiments, one inner monomer, e.g., 106A, and one outer monomer, e.g., 106B, dimerize (bind) together first to form a dimer, this interface being stronger, e.g., than the interface between first inner monomer 106A and second inner monomer 106C. Then, in some embodiments, two of the dimers come together to form a dimer of timers construct, having a comparatively weaker tetramer interface.

HIV IN 106 facilitates DNA fragment integration into a target DNA structure, presenting as a dimer of dimers with, in some embodiments, each dimer binding at least a portion of the polynucleotides for integration and catalyzing half site reactions jointly leading to full integration at a target site. In addition to containing nuclear localization signals (NLS), HIV IN 106 also relies on cellular proteins that are ubiquitous among eukaryotes, allowing it to be used in a wide range of host organisms. Among the different enzymes for achieving integration of transgenes, HIV-1 (IN) is an attractive candidate because of its high efficiency of insertion and robust activity. HIV-1 IN also achieves DNA integration without the risk of unpredictable deletions that can be seen with other systems using a non-homologous end joining (NHEJ) pathway.

The residues involved in the forming of the dimer interfaces in HIV IN 106 are located in the catalytic core domain (CCD) of the monomers. The dimer-dimer reaching interface is created by the N-terminal domain of one inner monomer, e.g., 106A, to the CCD of the opposing inner monomer, e.g., 106C. These two inner monomers perform the DNA integration reaction, with the outer monomers, e.g., 106B and 106D, performing a stabilizing role. The outer integrase monomers have their N-terminal domains free, and additional domains attached to these domains are relatively free to associate. In some embodiments, desired pairs of HIV IN monomers are produced as larger protein fusions with cleavable linkers that can be cut after protein production, e.g., by native proteases or by HIV-1 protease to free them up and allow them to associate.

In some embodiments, HIV IN 106 includes at least 85%, at least 90%, at least 95%, at least 96%, at least 97%, at least 98%, or at least 99% sequence identity with a wild-type HIV integrase, e.g., HIV-1 (IN). In some embodiments, HIV IN 106 maintains substantially equivalent DNA integration functionality as wild-type HIV integrase. In some embodiments, HIV IN 106 maintains substantially equivalent DNA integration functionality as HIV-1 (IN).

In some embodiments, HIV IN 106 includes a plurality of mutations relative to wild-type HIV integrases. In some embodiments, specific mutations were designed to bias binding of the different monomers to each other. In some embodiments, first inner monomer 106A includes mutations Y99D, K103E, K173E, K186E, or combinations thereof. In some embodiments, first inner monomer 106A includes functional equivalents to these mutations. In some embodiments, first inner monomer 106A includes SEQ. ID NO.: 1. In some embodiments, second inner monomer 106C includes mutations E96K, Y99E, K103E, V201H, D25K, E11K, or combinations thereof. In some embodiments, second inner monomer 106C includes functional equivalents to these mutations. In some embodiments, second inner monomer 106C includes SEQ. ID NO.: 2. In some embodiments, first outer monomer 106B includes mutations E87K, E96K, E152Q, K186Q, K215E, or combinations thereof. In some embodiments, first outer monomer 106B includes functional equivalents to these mutations. In some embodiments, first outer monomer 106B includes SEQ. ID NO.: 3. In some embodiments, second outer monomer 106D includes mutations E152Q, K173E, T174K, K186Q, I204D, or combinations thereof. In some embodiments, second outer monomer 106D includes functional equivalents to these mutations. In some embodiments, second outer monomer 106D includes SEQ. ID NO.: 4. In some embodiments, each of first outer monomer 106B and second outer monomer 106D include E152Q mutations. In some embodiments, each of first outer monomer 106B and second outer monomer 106D include K186Q mutations. In some embodiments, HIV IN 106 includes a mutation to reduce association of the HIV IN with LEDGF/p75, which normally gives the tetramer complex affinity to the target DNA structure and biases the insertion of DNA fragments to transcriptionally active regions of the chromosome. In some embodiments, HIV IN 106 includes a mutation at V165A of one or more monomers of HIV IN 106.

Still referring to FIG. 1, gene editing construct 102 includes one or more Transcription Activator Like Effectors (TALEs) 108. In some embodiments, recombinant enzyme 104 includes one or more TALEs 108. In some embodiments, TALEs 108 are bound to HIV IN 106. In some embodiments, at least two TALEs 108, e.g., a first TALE 108A and second TALE 108B, are bound to HIV IN 106. In some embodiments, TALEs 108 are bound to outer monomers, e.g., 106B, 106D, or combinations thereof. In some embodiments, TALEs 108 are bound to outer monomers, e.g., 106B, 106D, or combinations thereof, at the N-terminal regions of those outer monomers. In some embodiments, first TALE 108A is bound to first outer monomer 106B. In some embodiments, second TALE 108B is bound to second outer monomer 106D.

TALEs are natively found proteins that identify and bind to specific DNA segments to mark them. The DNA targeting of TALEs is based on two amino acids within repeating segments called repeat variable residues or RVDs. TALEs have a highly specific, rotationally decoupled linear search mechanism along DNA that grants them excellent accuracy even in densely packed DNA regions and especially in editing heterochromatin regions. Thus, TALEs represent a strong candidate for granting DNA site specificity to gene editing construct 102. TALEs can also be delivered in lentiviral vectors and can be encapsulated into HIV-1 inner capsids (as discussed in greater detail below), and thus will not affect activity of system 100 when targeting non-dividing cells.

By creating different versions of HIV IN monomers as described in the exemplary embodiments above, pairs of dimers were formed that each preferentially bind to form two specific heterodimers which would then form a heterotetramer, greatly increasing the likelihood of having both of the desired TALEs in the tetramer and on the outer monomers. In some embodiments, TALEs 108 are bound in gene editing construct 102, e.g., to HIV IN 106, by polypeptide linkers 110. In some embodiments, first TALE 108A is bound to first outer monomer 106B by a first polypeptide linker 110A. In some embodiments, first TALE 108A is bound to an N-terminal region of first outer monomer 106B by first polypeptide linker 110A. In some embodiments, second TALE 108B is bound to second outer monomer 106D by a second polypeptide linker 110B. In some embodiments, second TALE 106B is bound to an N-terminal region of second outer monomer 106D by second polypeptide linker 110B. In some embodiments, polypeptide linkers 110 are rigid. In some embodiments, polypeptide linkers 110 are flexible. In some embodiments, polypeptide linkers 110 are composed of glycine residues. In some embodiments, the polypeptide linkers 110 include repeats of GGGS domains, EAAAK domains, or combinations thereof. In some embodiments, the polypeptide linkers 110 include alternating repeats of GGGS domains, EAAAK domains, or combinations thereof. In some embodiments, the polypeptide linkers 110 include GGGS GGGS GGGS GGGS (SEQ. ID NO.: 5). Without wishing to be bound by theory, as the rigidity of polypeptide linkers 110 increases, so does the specificity of gene editing construct 102.

In some embodiments, one or more polynucleotides 112 are bound to gene editing construct 102. In some embodiments, polynucleotides 112 are any polynucleotide sequence (or transgene or DNA fragment) desired for insertion into genomic material of a cell. In some embodiments, polynucleotides 112 are bound to at least one of first inner monomer 106A and second inner monomer 106C. In some embodiments, HIV IN 106 binds selectively to the ends of a specific polynucleotide 112 (HIV IN 106 can be modified by making additional mutations, e.g., to allow for directional insertion into transgenes).

As discussed above, the remainder of gene editing construct 102, e.g., HIV IN 106 and TALEs 108 in conjunction with other lentiviral vector components, facilitate transport of polynucleotides 112 from an extracellular environment to the nuclear environment where it becomes available for insertion into the genome and subsequent replication. Gene editing constructs 102 consistent with embodiments of the present disclosure also enable such integration to occur with high site specificity and avoidance of off-target delivery. Previous attempts to alter certain proteins to increase their sequence specificity for DNA insertion have not been shown to yield the accuracy necessary to make them useful for clinical applications. In one example of how these weaknesses can affect the viability of treatment methods, a recent study attempted to use CRISPR to edit the genes of patients with sickle cell anemia. In this study, 2 of the 8 participants developed acute myeloid leukemia from unknown causes.

In some embodiments, a first TALE, e.g., 108A, is configured to bind a target DNA structure at a first region and a second TALE, e.g., 108B, is configured to bind the target DNA structure at a second region. In some embodiments, the first region and the second region are spaced apart on the target DNA structure by a predetermined number of base pairs. In some embodiments, the first region and the second region are spaced apart on the target DNA structure by between about 15 base pairs and about 25 base pairs. Upon binding of a first TALE and a second TALE at the target DNA structure, HIV IN 106 is localized with the catalytic region adjacent the desired insertion site of the genome.

In some embodiments, each TALE 108 targets a DNA structure segment greater than about 8 base pairs. In some embodiments, each TALE 108 targets a DNA structure segment between about 8 base pairs and about 31 base pairs. In some embodiments, first TALE 108A and second TALE 108B are of differing lengths. In some embodiments, first TALE 108A is longer than second TALE 108B. In some embodiments, first TALE 108A targets a DNA structure segment of about 30 base pairs. In these embodiments, larger TAL arrays will diffuse more slowly than smaller array. Without wishing to be bound by theory, the smaller array compresses the linker, e.g., polypeptide linker 110A, to HIV IN 106 and hinder the activity of HIV IN until the target DNA sequence is found. Once the smaller TAL array identifies the target DNA sequence, the larger TAL array stretches back to its own target DNA sequence, opening up the active site of HIV IN 106 and facilitating targeted DNA integration.

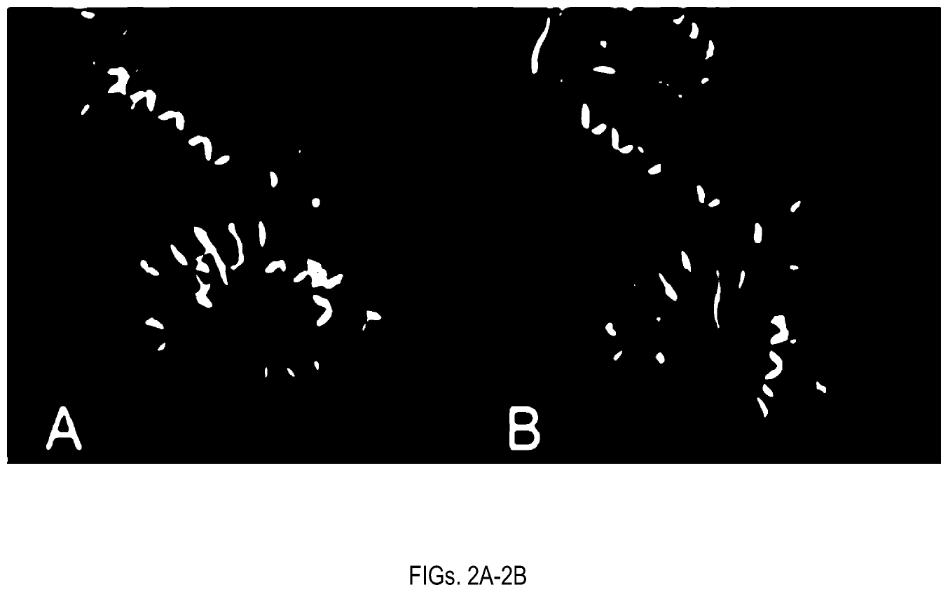

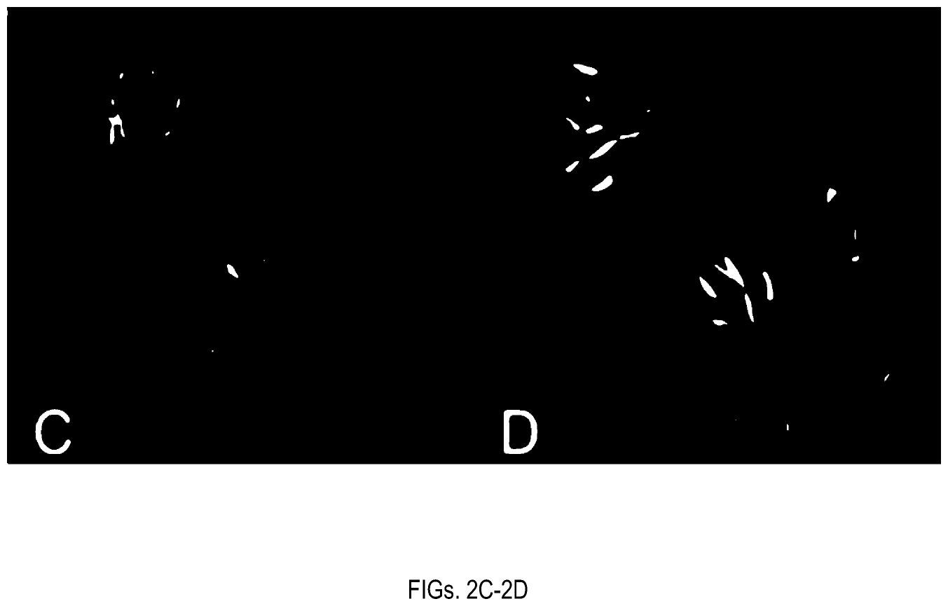

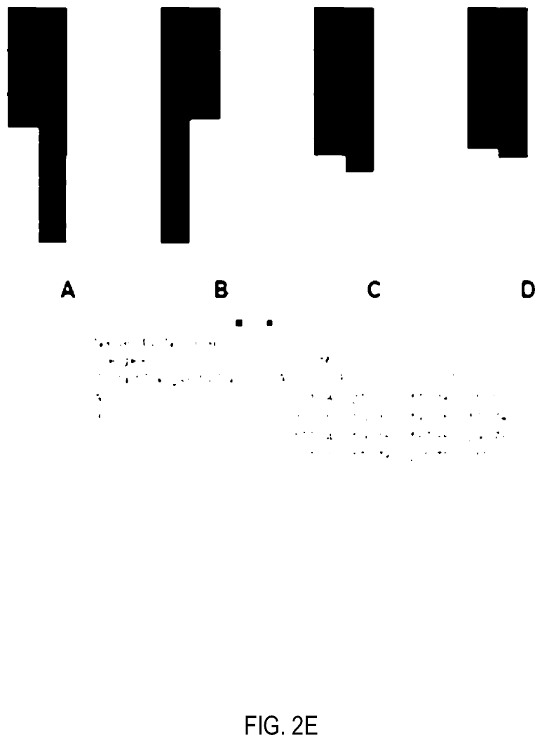

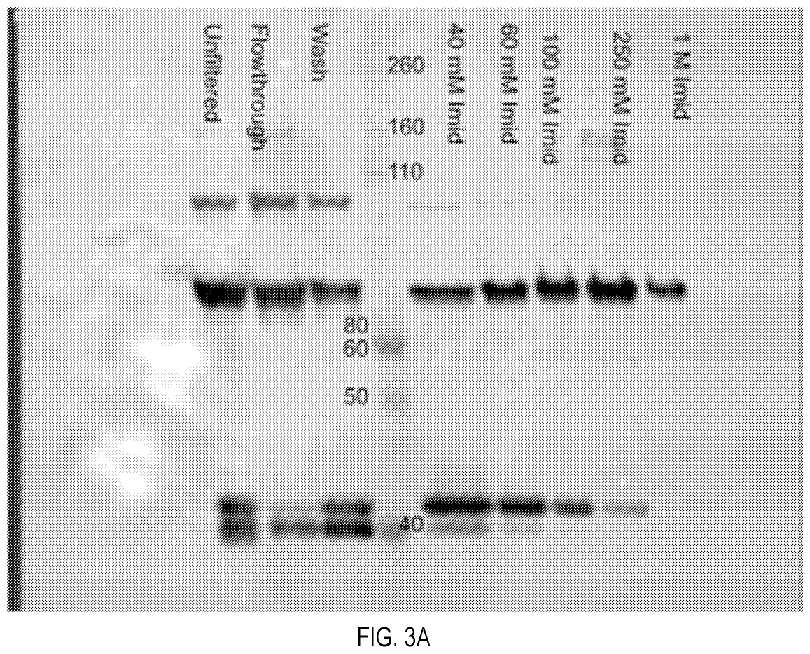

Referring now to FIGS. 2A-2E, the dimer interface and charge-swapping mutations on key residues of an exemplary embodiment of HIV IN 106 were selected and analyzed based on the examination of protein charge patches. In these exemplary embodiments, HIV IN 106 has complementary charge regions on the opposing monomers. As can be seen in the contact energy comparisons in FIGS. 2A-2D, A and B monomers have a much higher affinity for one another and less for any other monomer, with the same trend found for the C and D monomers. The non-polar binding regions of the integrase dimerization interface were left unmutated since HIV-1 IN, once formed into dimers, has excellent resistance to dissociation with a K_{d} of ~68 pM. To encourage the correct pairing of dimer pairs, another set of mutations was introduced, e.g., around K186, and its corresponding binding pair on the N-terminal region of the inner monomer of the second dimer pair, residues known to prevent tetramerization when mutated. The outer monomers B and D both carry the inactivating mutation, e.g., E152Q, which prevents them from performing the integration reaction on their own and also contain the mutation that prevents them from being the inner monomers in the tetramer structure, e.g., K186Q. A mutation was entered to reduce binding to LEDGF/p75, e.g., at V165A, which normally gives the tetramer complex affinity to DNA and biases the insertion of DNA to transcriptionally active regions of the chromosome.

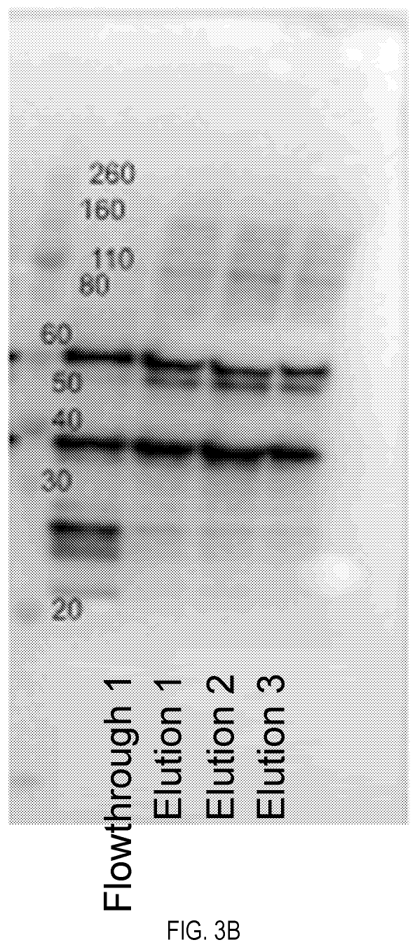

Referring now to FIGS. 3A-3B, after creating the four exemplary monomeric mutants with site-directed mutagenesis, sfGFP was fused to IN A, a His7 tag was fused to IN B, mCherry was fused to IN C, and a strep tag was fused to IN D. First, a plasmid containing IN A and IN B was expressed in BL21. The protein was resuspended with lysis buffer containing 1 M Urea and only 0.1 M NaCl. Purification was performed using a His nickel column with increasing concentrations of imidazole to reduce contamination by potential homodimers. Because of the addition of GFP to one of the monomers and not the other, there is a size difference between IN A (58.9 kDa) and IN B (33.1 kDa); this is also true for IN C (59.1 kDa) and IN D (33.5 kDa) as shown in the Western blots in FIG. 3A. In the GFP Western blot of AB samples, the Western blot clear dimer bands were identified at ~90 kDa as well as monomer bands lower down. Additionally, to verify the presence of GFP in the observed bands, a Western blot was performed using an anti-GFP antigen. The results of the SDS-PAGE and Western blots showed a single band in the 1 M imidazole elutions at about 90 kDa, which is a band expected for the desired heterodimer with no observable homodimer bands (~66 kDa) there or in other elutions. The existence of a dimer band in the SDS-PAGE demonstrates a strong binding interface, expected given that HIV IN does maintain dimers in the denaturing conditions used (SDS and beta-mercaptoethanol). The same procedure was repeated for IN C and D with filtration performed with a column of strep-tactin XT 4 flow resin. The results can be seen in FIG. 3B, which shows an mCherry Western blot showing monomer bands at ~60 kDa, what seems to be secondary transcription products at <40 kDa (this is likely full mCherry product as methionine was not removed during fusion), and dimer bands can also be seen at ~90 kDa. In this sample, weaker binding resulted in less dimer being observed under the denaturing condition, which is to be expected since the binding energy of A and B monomer should be significantly stronger than that between C and D.

Referring again to FIG. 1, in some embodiments, gene editing construct 102 is encapsulated in a lentiviral capsid 114. In some embodiments, system 100 is incorporated into a composition for administration in a target organism, e.g., a patient. In some embodiments, the composition is formulated for any desired route of administration, e.g., intravenous, nasal, topical, oral, inhalation, etc., or combinations thereof. In some embodiments, system 100 is incorporated into target cells via suitable transformation processes, e.g., chemical transformation of cells in vitro. In some embodiments, system 100 is incorporated into target cells via a suitable biolistic particle delivery system, e.g., gene gun for use with plant cells. In some embodiments, the composition includes an effective amount of gene editing construct 102, e.g., sufficient to effectuate the desired editing of a target organism's genome and achieve a desired treatment outcome. In some embodiments, the composition includes one or more additional active ingredients, pharmaceutically acceptable adjuvants, diluents, excipients, carriers, or combinations thereof.

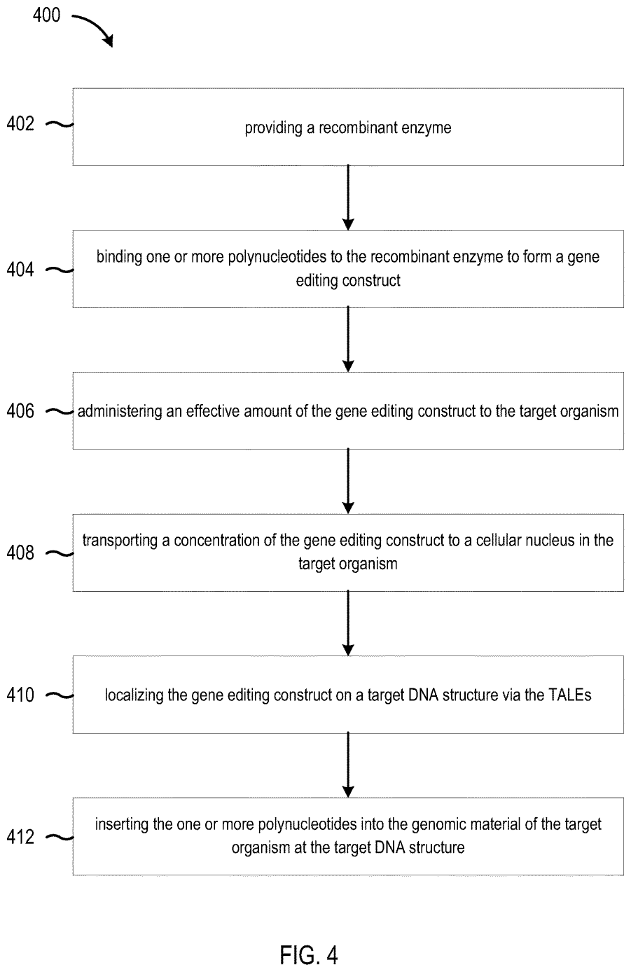

Referring now to FIG. 4, some embodiments of the present disclosure include a method 400 of editing genomic material, e.g., in a patient, cell, etc. As discussed above, in some embodiments, method 400 edits genomic material in an effort to provide a therapeutic effect to the cell/target organism. In some embodiments, method 400 edits genomic material in an effort to increase production by the cell/target organism of one or more desired compounds, e.g., increased chemical production. At 402, a recombinant enzyme is provided. As discussed above, in some embodiments, the recombinant enzyme includes an HIV IN and two or more TALEs bound to the HIV IN. In some embodiments, a first TALE is bound to a first outer monomer in the HIV IN. In some embodiments, a second TALE is bound to a second outer monomer in the HIV IN. In some embodiments, the TALEs are bound in the gene editing construct, e.g., at HIV IN, by polypeptide linkers. In some embodiments, the first TALE is bound to an N-terminal region of the first outer monomer by a first polypeptide linker. In some embodiments, the second TALE is bound to an N-terminal region of the second outer monomer by a second polypeptide linker. In some embodiments, the first TALE and the second TALE are of differing lengths. In some embodiments, the first TALE is longer than the second TALE. In some embodiments, the polypeptide linkers are rigid. In some embodiments, the polypeptide linkers are flexible. In some embodiments, the polypeptide linkers are composed of glycine residues. In some embodiments, the polypeptide linkers include repeats of GGGS domains, EAAAK domains, or combinations thereof. In some embodiments, the polypeptide linkers include alternating repeats of GGGS domains, EAAAK domains, or combinations thereof. In some embodiments, the polypeptide linkers include GGGS GGGS GGGS GGGS (SEQ. ID NO.: 5).

In some embodiments, the HIV IN includes at least 85%, at least 90%, at least 95%, at least 96%, at least 97%, at least 98%, or at least 99% sequence identity with a wild-type HIV integrase, e.g., HIV-1 (IN). In some embodiments, the HIV IN maintains substantially equivalent DNA integration functionality as wild-type HIV integrase. In some embodiments, the HIV IN maintains substantially equivalent DNA integration functionality as HIV-1 (IN).

In some embodiments, the HIV IN includes a plurality of mutations relative to wild-type HIV integrases. In some embodiments, specific mutations were designed to bias binding of the different monomers to each other. In some embodiments, the first inner monomer includes mutations Y99D, K103E, K173E, K186E, or combinations thereof. In some embodiments, the first inner monomer includes SEQ. ID NO.: 1. In some embodiments, the second inner monomer includes mutations E96K, Y99E, K103E, V201H, D25K, E11K, or combinations thereof. In some embodiments, the second inner monomer includes SEQ. ID NO.: 2. In some embodiments, the first outer monomer includes mutations E87K, E96K, E152Q, K186Q, K215E, or combinations thereof. In some embodiments, the first outer monomer includes SEQ. ID NO.: 3. In some embodiments, the second outer monomer includes mutations E152Q, K173E, T174K, K186Q, I204D, or combinations thereof. In some embodiments, second outer monomer 106D includes SEQ. ID NO.: 4. In some embodiments, each of the first outer monomer and the second outer monomer include E152Q mutations. In some embodiments, each of the first outer monomer 106B and the second outer monomer include K186Q mutations. In some embodiments, the HIV IN includes a mutation to reduce association of the HIV IN with LEDGF/p75, which normally gives the tetramer complex affinity to the target DNA structure and biases the insertion of DNA fragments to transcriptionally active regions of the chromosome. In some embodiments, HIV IN includes a mutation at V165A of one or more monomers of the HIV IN.

Still referring to FIG. 4, in some embodiments, method 400 includes binding 404 one or more polynucleotides to the recombinant enzyme to form a gene editing construct, e.g., gene editing construct 102 discussed above. In some embodiments, the polynucleotides are bound to at least one of the first inner monomer and the second inner monomer of HIV IN. In some embodiments, the gene editing construct is encapsulated in a lentiviral capsid. In some embodiments, the gene editing construct is part of a lentiviral vector.

At 406, an effective amount of the gene editing construct is administered, e.g., to a target organism or patient. As discussed above, in some embodiments, the gene editing construct is administered as part of a composition formulated for any desired route of administration, e.g., intravenous, nasal, topical, oral, inhalation, etc., or combinations thereof. At 408, a concentration of the gene editing construct is transported to a cellular nucleus. At 410, the gene editing construct is localized on a target DNA structure via the TALEs. In some embodiments, the first TALE is configured to bind a target DNA structure at a first region and the second TALE is configured to bind the target DNA structure at a second region. In some embodiments, the first region and the second region are spaced apart on the target DNA structure by a predetermined number of base pairs. In some embodiments, the first region and the second region are spaced apart on the target DNA structure by between about 15 base pairs and about 25 base pairs. Upon binding of the first TALE and the second TALE at the target DNA structure, the HIV IN is localized with the catalytic region adjacent the desired insertion site. At 412, the polynucleotides are inserted via the HIV IN at the target DNA structure, i.e., into the genomic material of the target.

Systems and methods of the present disclosure advantageously utilize recombinant enzymes including modified HIV-1 integrase tetramers fused to two separate TALEs for use as site specific gene editors, capable of editing eukaryotic cells via standard transformation processes. HIV-1 integrase works reliably in non-dividing cells, is resistant to degradation and adverse conditions, exhibits low immunogenicity, and further does not rely on non-ubiquitous cellular coenzymes/repair mechanisms, and has been shown to work in organisms as diverse as Saccharomyces cerevisiae. The modified HIV-1 integrases can form pre-integration gene editing constructs with polynucleotides to simplify construction and delivery of the constructs.

The HIV IN tetramers according to embodiments of the present disclosure include mutated monomeric subunits, 2 sets of 2 mutated variants that pair specifically to each other using a charged swapping approach for greater binding affinity associated with matching the correct pairing of integrases together compared to a much lower binding affinity with non-matching pairs to facilitate production of the correct monomer pairings. These mutations are applied along both the monomer-monomer interface and the dimer-dimer interface, with additional inactivating mutations to outer monomers to ensure activation/deactivation of the desired monomers.

The presence of the two TALEs grants the recombinant enzyme full sequence specificity and prevents non-specific interactions with a target DNA structure. The modified HIV-1 integrase monomers ensure proper binding of TALEs to the outer-most monomers and arrangement of the TALEs relative to the catalytic region of the HIV IN, granting the overall construct swift, high-efficiency, site-specific targeting and integration of DNA fragments into the target DNA structure. These gene editing constructs can be used both on their own and/or integrated into existing lentiviral vectors to allow for the efficient and accurate insertion of transgenes into desired genomic locations with a focus on accuracy as measured by achieving virtually no off-target mutations or indels.

Although the invention has been described and illustrated with respect to exemplary embodiments thereof, it should be understood by those skilled in the art that the foregoing and various other changes, omissions and additions may be made therein and thereto, without parting from the spirit and scope of the present invention.

What is claimed is:

1. A recombinant enzymatic gene editing system, comprising:
a recombinant enzyme, the enzyme including:
an HIV integrase (HIV IN) tetrameric structure; and
two or more Transcription Activator Like Effectors (TALEs) bound to the HIV IN tetramer,
one or more polynucleotides bound to the HIV IN to form a gene editing construct.

2. The system according to claim 1, wherein the HIV IN includes four monomeric subunits including a first inner monomer and a second inner monomer and a first outer monomer and a second outer monomer, wherein at least two of the monomeric subunits have a TALE bound to N-terminal domains thereof.

3. The system according to claim 2, wherein a first TALE is bound to the first outer monomer of the HIV IN by a first polypeptide linker, and a second TALE is bound to the second outer monomer of the HIV IN by a second polypeptide linker.

4. The system according to claim 3, wherein the first polypeptide linker and the second polypeptide linker are flexible, rigid, or combinations thereof.

5. The system according to claim 3, wherein the first and second TALEs are of differing lengths.

6. The system according to claim 3, wherein the first TALE is configured to bind a target DNA structure at a first region and the second TALE is configured to bind the target DNA structure at a second region, wherein the first region and the second region are spaced apart on the target DNA structure by between about 15 base pairs and about 25 base pairs.

7. The system according to claim 3, wherein the HIV IN includes a wild-type HIV IN having:
a mutation to reduce HIV IN association with LEDGF/p75;
E152Q mutations to each of the first outer monomer and the second outer monomer;
K186Q mutations to each of the first outer monomer and the second outer monomer,
or combinations thereof.

8. The system according to claim 1, wherein the gene editing construct is provided in a lentiviral capsid, as part of a lentiviral vector, or combinations thereof.

9. A method of editing genomic material in a target organism, comprising:
providing a recombinant enzyme, the enzyme including:
an HIV integrase (HIV IN) tetrameric structure; and
two or more Transcription Activator Like Effectors (TALEs) bound to the HIV IN tetramer,
binding one or more polynucleotides to the recombinant enzyme to form a gene editing construct;
administering an effective amount of the gene editing construct to the target organism;
transporting a concentration of the gene editing construct to a cellular nucleus in the target organism;
localizing the gene editing construct on a target DNA structure via the TALEs; and
inserting the one or more polynucleotides into the genomic material of the target organism at the target DNA structure.

10. The method according to claim 9, wherein:
the HIV IN includes four monomeric subunits including a first inner monomer and a second inner monomer and a first outer monomer and a second outer monomer, wherein at least two of the monomeric subunits have a TALE bound to N-terminal domains thereof, and
a first TALE is bound to a first outer monomer of the HIV IN by a first polypeptide linker, and a second TALE is bound to a second outer monomer of the HIV IN by a second polypeptide linker.

11. The method according to claim 10, wherein the first polypeptide linker and the second polypeptide linker are flexible, rigid, or combinations thereof.

12. The method according to claim 10, wherein the first TALE is longer than the second TALE.

13. The method according to claim 10, wherein the first TALE is configured to bind the target DNA structure at a first region and the second TALE is configured to bind the target DNA structure at a second region, wherein the first region and the second region are spaced apart on the target DNA structure by between about 15 base pairs and about 25 base pairs.

14. The method according to claim 10, wherein the HIV IN includes a wild-type HIV IN having:
a mutation to reduce HIV IN association with LEDGF/p75;
E152Q mutations to each of the first outer monomer and the second outer monomer;
K186Q mutations to each of the first outer monomer and the second outer monomer,
or combinations thereof.

15. The method according to claim 9, wherein the gene editing construct is provided in a lentiviral capsid, as part of a lentiviral vector, or combinations thereof.

16. A recombinant enzymatic gene editing system, comprising:
a recombinant enzyme, the enzyme including:
an HIV integrase (HIV IN) tetramer including a first inner monomer and a second inner monomer and a first outer monomer and a second outer monomer, wherein the first inner monomer and the second inner monomer are bound to each other and the first outer monomer is bound to the first inner monomer and the second outer monomer is bound to the second inner monomer; and
a first Transcription Activator Like Effector (TALEs bound to an N-terminal region of the first outer monomer via a first polypeptide linker and a second TALE bound to an N-terminal region of the second outer monomer via a second polypeptide linker,
one or more polynucleotides bound to at least one of the first inner monomer and the second inner monomer to form a gene editing construct, and
a lentiviral capsid encapsulating the gene editing construct.

17. The system according to claim 16, wherein the first polypeptide linker and the second polypeptide linker include GGGS GGGS GGGS GGGS (SEQ. ID NO.: 5).

18. The system according to claim 16, wherein the first TALE is longer than the second TALE.

19. The system according to claim 18, wherein the first TALE targets a DNA structure segment of about 30 base pairs.

20. The system according to claim 16, wherein the HIV IN tetramer includes a wild-type HIV IN having the following mutations:
for the first inner monomer, Y99D, K103E, K173E, and K186E;
for the second inner monomer, E96K, Y99E, K103E, V201H, D25K, and E11K;
for the first outer monomer, E87K, E96K, E152Q, K186Q, and K215E;
for the second outer monomer, E152Q, K173E, T174K, K186Q, and I204D; and
a mutation to reduce HIV IN association with LEDGF/p75.


SEQUENCE LISTING (5 sequences)

SEQ ID NO: 1 - protein, 290 aa, synthetic construct
MGFLDGIDKAQEEHEKYHSNWRAMASDFNLPPVVAKEIVASCDKCQLKGEAMHGQVDCSPGIWQLDCTHLEGKVILVAVHVASGYIEAEVIPAETGQETADFLLELAGRWPVKTVHTDNGSNFTSTTVKAACWWAGIKQEFGIPYNPQSQGVIESMNKELKKIIGQARDQAEHLETAVQMAVFIHNFERKGGIGGYSAGERIVDIIATDIQTKELQKQITKIQNFRVYYRDSRDPVWKGPAKLLWKGEGAVVIQDNSDIKVVPRRKAKIIRDYGKQMAGDDCVASRQDED

SEQ ID NO: 2 - protein, 290 aa, synthetic construct
MGFLDGIDKAQEKHEKYHSNWRAMASKFNLPPVVAKEIVASCDKCQLKGEAMHGQVDCSPGIWQLDCTHLEGKVILVAVHVASGYIEAEVIPAETGQKTAEFLLELAGRWPVKTVHTDNGSNFTSTTVKAACWWAGIKQEFGIPYNPQSQGVIESMNKELKKIIGQARDQAEHLKTAVQMAVFIHNFKRKGGIGGYSAGERIHDIIATDIQTKELQKQITKIQNFRVYYRDSRDPVWKGPAKLLWKGEGAVVIQDNSDIKVVPRRKAKIIRDYGKQMAGDDCVASRQDED

SEQ ID NO: 3 - protein, 288 aa, synthetic construct
FLDGIDKAQEEHEKYHSNWRAMASDFNLPPVVAKEIVASCDKCQLKGEAMHGQVDCSPGIWQLDCTHLEGKVILVAVHVASGYIEAKVIPAETGQKTAYFLLKLAGRWPVKTVHTDNGSNFTSTTVKAACWWAGIKQEFGIPYNPQSQGVIQSMNKELKKIIGQARDQAEHLKTAVQMAVFIHNFQRKGGIGGYSAGERIVDIIATDIQTKELQEQITKIQNFRVYYRDSRDPVWKGPAKLLWKGEGAVVIQDNSDIKVVPRRKAKIIRDYGKQMAGDDCVASRQDED

SEQ ID NO: 4 - protein, 288 aa, synthetic construct
FLDGIDKAQEEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTVHTDNGSNFTSTTVKAACWWAGIKQEFGIPYNPQSQGVIQSMNKELKKIIGQARDQAEHLEKAVQMAVFIHNFQRKGGIGGYSAGERIVDIDATDIQTKELQKQITKIQNFRVYYRDSRDPVWKGPAKLLWKGEGAVVIQDNSDIKVVPRRKAKIIRDYGKQMAGDDCVASRQDED

SEQ ID NO: 5 - protein, 16 aa, synthetic construct
GGGSGGGSGGGSGGGS